(19) United States
(12) Patent Application Publication
(10) Pub. No.: US 2020/0054480 A1
(43) Pub. Date: Feb. 20, 2020

(54) CONDOM WITH WRAPPING ADHESIVE FLANGES

(71) Applicant: John Ing Ching TANG, Sibu (MY)

(72) Inventor: John Ing Ching TANG, Sibu (MY)

(21) Appl. No.: 16/610,054

(22) PCT Filed: Apr. 29, 2018
(86) PCT No.: PCT/IB2018/052965

§ 371 Date: Oct. 31, 2019

(30) Foreign Application Priority Data
May 2, 2017 (MY) PI 2017701531

(51) Int. Cl. A61F 6/04

(52) U.S. Cl. CPC A61F 6/04; A61F 2006/047

(57) ABSTRACT

A condom comprises a distal non-adhesive tubular portion 1 and a proximal circumferentially outstretched adhesive flange 2. The tubular portion 1 covers the distal part of the penis, whereas the adhesive flange 2 wraps around and sticks onto the proximal part of the penis to prevent slippage and leakage of the condom. The folds created by the flange (2A and 2B) adhering to itself are used as holding tags to ease the condom's removal. The condom may be produced by sealing and cutting two thermoplastic films to create a pouch with a substantially hat shape seam, and two adhesive flanges (2A and 2B) extending from the seam to emulate the above configuration. The adhesive coating may be on the external surface of the flanges and the condom is inverted outside-in to emulate the above configuration.

### TECHNICAL FIELD

The objective of the present invention is related to sexual barriers for the prevention of fertilizing spermatozoa and sexually transmitted diseases during sexual intercourse. Particularly it relates to the protection of male organ to prevent exchange of body fluid by a thin flexible walled condom with adhesive.

### BACKGROUND ART

Male latex condom is the commonest used barrier methods for the prevention of unintended pregnancies and sexually transmitted infections. However, it suffers from slippage/leakage problem whenever penis is not tumescent enough to keep the condom stretched, which happens not only after ejaculation, but often along the course of sexual act, both in situ and ex situ.

Multiple prior art have attempted to solve the above problems by coating adhesive on the inner surface of condom in order to stick it onto penis. An example is shown in prior art CN203107378U, which discloses a condom wherein the internal surface of its open end is coated with adhesive. However, it does not elaborate on how the adhesive coating can be achieved or how the user can put it on. Indeed, the addition of adhesive to the internal surface of a tubular, flimsy and flexible condom remains a difficult challenge in both its manufacture and deployment.

U.S. Pat. No. 4,638,790 discloses a condom in which its hood, initially in a rolled-up configuration, has an outer member which transfers adhesive onto the inner surface of the condom as the hood is unrolled onto the penis. The outer member is removed after the condom is in place. The main problem with such a configuration is that the user's hands are in direct contact with the adhesive surface during unrolling of the condom on the penis, thereby affecting the integrity of pressure sensitive adhesive. Furthermore, the invention does not disclose any means, such as holding tags, to ease removal of the condom.

In a patent granted to Attila (U.S. Pat. No. 7,963,285), it is provided a rolled condom with adhesive on the inner surface supported by a cylindrical releasing strip. The cylindrical releasing strip extends beyond the open end of condom and flares out circumferentially to form a graspable deployment rim/ring. The condom together with releasing strip within it is rolled up during assembly. It is unrolled onto penis by pulling the releasing strip proximally using the rim/ring. The problem with such a design is that while donning a rolled non-adhesive condom by direct unrolling action is relatively easy, donning a rolled adhesive condom by pulling on the releasing strip is more difficult as a lot more force is required at unrolling an adhesive entity let alone pulling it. Moreover, the manufacturing of rolled adhesive condom that involves application of relatively non-elastic releasing strip on an adhesive coated elastic condom followed by rolling is technically difficult.

There is therefore a need for an adhesive condom that is cheap to produce and easy to be deployed and removed by users for the prevention of unintended pregnancy and sexually transmitted infections.

### SUMMARY OF THE INVENTION

In accordance with the present invention there is provided an adhesive condom that is to be worn by a male person to prevent the transmission of infections and sperm during sexual intercourse.

The adhesive condom has two parts: a distal non-adhesive tubular portion at the closed end and a proximal adhesive flange at the open end. While the tubular portion dons on the distal part of penis, the flange adheringly wraps around the proximal part of penis so that it will not slip regardless of penis's degree of tumescence.

As discussed above, the main difficulty in the production of adhesive condom is in the coating of adhesive on the internal surface of the flimsy condom. In the present invention, this is overcome by having a circumferentially outstretched flange at the open end of condom, so that coating of adhesive can easily be performed on the now exposed internal surface of the flange, supported by releasing paper. The coating of adhesive may also be performed on the external surface of the flange, and the condom is inverted outside-in to internalize the flange's adhesive surface to be deployed on the penis.

The deployed condom thus has two distinctive tubular parts: firstly, the tubular portion at the closed end of condom, and secondly, the tubular part formed by wrapping of the flange on the penis. Therefore, in contrast with other adhesive condoms, in the present invention, both the tubular portion and the flange make up the condom that covers the penis. In particular, the proximal part (the flange) of the condom is non-tubular to start with, but only becomes tubular after its deployment onto the penis as it wraps around and conforms to the tubular shape of the penis, thus creating a novel condom comprising a distal tubular portion and a proximal outstretched adheringly wrapping flange.

It is another objective of the present invention to create one or more holding tags by adhering the “redundant flange”, the part that is not adhering to penis, onto itself. The holding tags is then used as handles to facilitate the removal of the adhesive condom.

The other objective of the present invention is to make an adhesive condom from sealing and cutting two thermoplastic films to create a pouch with a substantially hat shape seam (an inverted U shape seam with outstretched seams from both ends of the inverted U shape seam) with two flanges extending from the seam, wherein the flanges are coated with adhesive on the internal surfaces, to give a configuration of the condom with the tubular portion (formed by the inverted U shape seam) with the outstretched adhesive flange as described above. The adhesive coating may also be coated on the external surface of the flanges, whereby the condom is inverted outside-in to internalize the adhesive surfaces to emulate the condom as described above.

To ease handling at manufacture and deployment, the tubular portion and the adhesive flanges may be appropriately backed by more rigid carrier films, and likewise the adhesive surfaces by releasing papers.

### BRIEF DESCRIPTION OF THE DRAWINGS

FIG. 1 is a top view of a preferred embodiment of the present invention wherein the open end of the condom comprises a circumferentially outstretched flange.

FIG. 2 is a bottom view of FIG. 1;

FIG. 3 is a front view of the condom, as in FIGS. 1 and 2 duly, deployed onto the penis;

FIG. 4 is a perspective view of another preferred embodiment wherein the tubular portion and its two flanges are manufactured by sealing and cutting two thermoplastic films;

FIG. 5 is a front view of the condom, as in FIG. 4, duly deployed onto the penis.

### DETAILED DESCRIPTION OF PREFERRED EMBODIMENTS

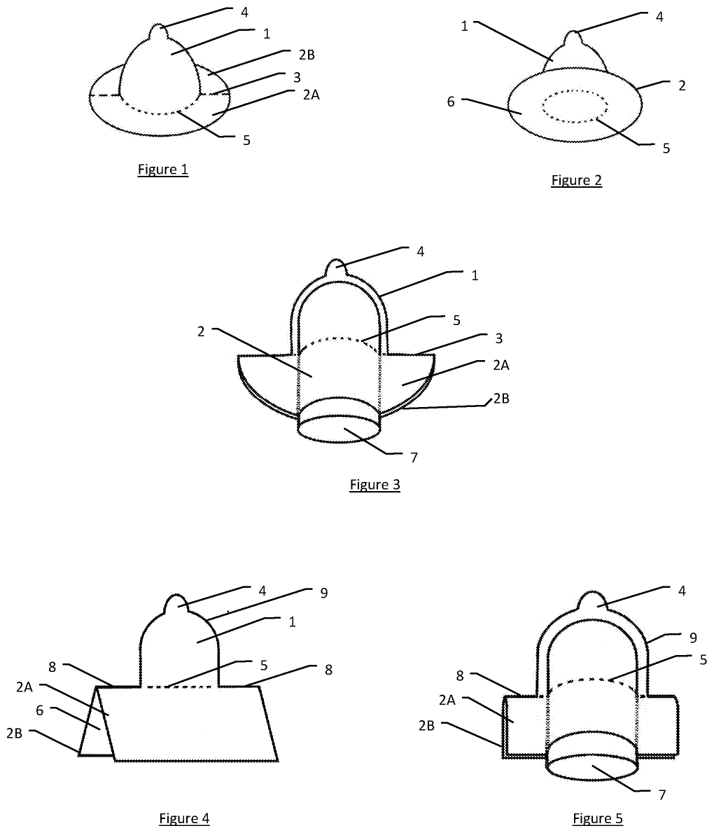

In a preferred embodiment, the adhesive condom (FIG. 1) has a tubular portion 1 at the closed end and a circumferentially outstretched flange 2 (consists of anterior section 2A and posterior section 2B) at the open end. The flange 2 has adhesive coating, demarcated by adhesive margin 5, on the internal (or the lower) surface 6 (FIG. 2), and has an imaginary folding line 3 where the flange is folded into two halves (2A and 2B) at deployment. During deployment (FIG. 3), with or without the help of lubricant, the tubular portion 1 is donned over the distal part of the penis while both anterior flange 2A and posterior flange 2B are folded downward to adhere onto the proximal part of the penis 7 and onto each other (2A and 2B). The flange 2 now assumes a tubular shape in continuum with the tubular portion 1, with one or more protruding folds from the flange-to-flange adhesions (2A and 2B, FIG. 3). There are thus flange-to-flange (2a and 2B) and flange-to-penis (2 and 7) adhesions to provide a secure non-slip and non-spill deployment of the condom, even if the penis is not tumescent. Since the flange 2 is thin and flexible, the resultant protruding folds will not adversely interfere with sexual intercourse.

Because the tubular portion 1 is adhesive free, as demarcated by the adhesive coating margin 5 (FIG. 3), it spares the sensitive glans penis from possible painful removal of the adhesive flange 2 from the penis. In other words, the adhesive in the present embodiment is mainly in contact with the less sensitive skin of the penile shaft, not the sensitive glans penis.

The other benefit of the present invention is that the folds created by the flange-to-flange (2A and 2B) adhesions can be used as holding tags to enable easy removal of the condom.

In another embodiment, there is a seminal receptacle 4 at the closed end of the condom.

In yet another embodiment (FIG. 4), the condom is made by sealing and cutting two flexible thermoplastic films to create a pouch with a substantially inverted U shape seam 9 with extending seams 8 arising from both ends of the inverted U shape seam 9 (thus creating a substantially hat shape seam), and two flanges (2A and 2B) extending from the hat shape seam. The internal surfaces 6 of the two flanges (2A and 2B) are coated with adhesive. The U shape seam 9 forms the tubular portion 1 of the condom while its open end and the two extended seams 8 join the two adhesive flanges 2A and 2B to give an outstretched flanges' configuration similar to the embodiment as shown in FIG. 1. Likewise, at deployment (FIG. 5) the condom will have the similar configuration as described above (FIG. 3).

To ease manufacture processes, the adhesive may be coated on the external surfaces of the flanges 2A and 2B (FIG. 4). The tubular portion 1 is then inverted to turn the flanges (2A and 2B) outside-in to internalize the adhesive surfaces prior to deployment.

To make the flimsy tubular portion 1 and the adhesive flange 2 more manageable during production and deployment, both may be supported by more rigid carrier films and/or releasing paper, both of which are to be removed during the condom's deployment on the penis 7.

1. A condom that is divided into distal and proximal parts, wherein
a. the distal part of the condom (towards closed end) comprises a tubular portion that covers the distal part of the penis;
b. the proximal part of the condom (towards open end) comprises a circumferentially outstretched flange, wherein
i. the said flange has adhesive coating on its internal or lower surface;
ii. the said adhesive flange sticks onto the proximal part of the penis and onto itself to create a tubular covering, in continuum with the tubular portion, on the penis;
iii. folds formed by the flange adhering onto itself are used as holding tags for the removal of the condom.

2. The condom as in claim 1, wherein the condom has a tubular portion and two adhesive flanges. It is made by sealing and cutting two thermoplastic films to create a pouch with a substantially hat shape seam, wherein there are two adhesive flanges extending from the said seam to emulate the configuration of claim 1.

3. The condom as in the claims above, wherein the adhesive coating is on the external or upper surface of the flanges, and the condom is inverted to turn the adhesive surfaces outside-in to emulate the configuration of claim 1.

4. The condom as in the claims above, wherein the closed end of the condom has a protrusion that serves as a seminal receptacle.

5. The condom as in the claims above, wherein the tubular portion and/or the adhesive flanges are supported by carrier films and/or releasing papers.
